SEQUENCE LISTING (37 sequences)

SEQ ID NO: 1 - protein, 15 aa, unknown
  GSK3a
LRYFFYSSGEKKDEL

SEQ ID NO: 2 - protein, 15 aa, unknown
  GSK3b
LRYFFYSSGEKKDEV

SEQ ID NO: 3 - protein, 14 aa, unknown
  CDK4
LMDVCATSRTDREI

SEQ ID NO: 4 - protein, 14 aa, unknown
  CDK6
LFDVCTVSRTDRET

SEQ ID NO: 5 - protein, 13 aa, unknown
  TGFbRII
FLTAEERKTELGK

SEQ ID NO: 6 - protein, 13 aa, unknown
  ACTRIIA
FIGAEKRGTSVDV

SEQ ID NO: 7 - protein, 13 aa, unknown
  ACTRIIB
FIAAEKRGSNLEV

SEQ ID NO: 8 - protein, 13 aa, unknown
  ALK1
FIASDMTSRNSST

SEQ ID NO: 9 - protein, 13 aa, unknown
  ALK2
FIASDMTSRHSST

SEQ ID NO: 10 - protein, 13 aa, unknown
  ALK3
FIAADIKGTGSWT

SEQ ID NO: 11 - protein, 13 aa, unknown
  ALK4
FIAADNKDNGTWT

SEQ ID NO: 12 - protein, 14 aa, unknown
  BMPR-II
FIVGDERVTADGRM

SEQ ID NO: 13 - protein, 14 aa, unknown
  ERK1
IRDILRAPTLEAMR

SEQ ID NO: 14 - protein, 14 aa, unknown
  ERK2
INDIIRAPTIEQMK

SEQ ID NO: 15 - protein, 23 aa, unknown
  ERK3
VFEILGPSGSQLTDDVGSLTELN

SEQ ID NO: 16 - protein, 15 aa, unknown
  JNK1
LLNVFTPQKSLEEFQ

SEQ ID NO: 17 - protein, 15 aa, unknown
  JNK2
LLNVFTPQKTLEEFQ

SEQ ID NO: 18 - protein, 9 aa, unknown
  MOD_RES (1)...(0): Acyl
SSGEKKDEL

SEQ ID NO: 19 - protein, 12 aa, unknown
  MOD_RES (1)...(0): Hexyl
GFYSSGEKKDEL

SEQ ID NO: 20 - protein, 10 aa, unknown
  MOD_RES (1)...(0): Stearyl
GSSGEKKDEV

SEQ ID NO: 21 - protein, 11 aa, unknown
  MOD_RES (1)...(0): Hexyl
GSATSRTDREI

SEQ ID NO: 22 - protein, 10 aa, unknown
  MOD_RES (1)...(0): Stearyl
GATSRTDREI

SEQ ID NO: 23 - protein, 10 aa, unknown
  MOD_RES (1)...(0): Stearyl
GTVSRTDRET

SEQ ID NO: 24 - protein, 10 aa, unknown
  MOD_RES (1)...(0): Stearyl
GEERKTELGK

SEQ ID NO: 25 - protein, 10 aa, unknown
  MOD_RES (1)...(0): Stearyl
GEKRGTSVDV

SEQ ID NO: 26 - protein, 10 aa, unknown
  MOD_RES (1)...(0): Stearyl
GEKRGSNLEV

SEQ ID NO: 27 - protein, 10 aa, unknown
  MOD_RES (1)...(0): Stearyl
GDMTSRNSST

SEQ ID NO: 28 - protein, 10 aa, unknown
  MOD_RES (1)...(0): Stearyl
GDMTSRHSST

SEQ ID NO: 29 - protein, 10 aa, unknown
  MOD_RES (1)...(0): Stearyl
GDIKGTGSWT

SEQ ID NO: 30 - protein, 10 aa, unknown
  MOD_RES (1)...(0): Stearyl
GDNKDNGTWT

SEQ ID NO: 31 - protein, 11 aa, unknown
  MOD_RES (1)...(0): Stearyl
GDERVTADGRM

SEQ ID NO: 32 - protein, 9 aa, unknown
  MOD_RES (1)...(0): Acyl
ERVTADGRM

SEQ ID NO: 33 - protein, 9 aa, unknown
  MOD_RES (1)...(0): Acyl
RAPTLEAMR

SEQ ID NO: 34 - protein, 9 aa, unknown
  MOD_RES (1)...(0): Acyl
RAPTIEQMK

SEQ ID NO: 35 - protein, 9 aa, unknown
  MOD_RES (1)...(0): Acyl
DVGSLTELN

SEQ ID NO: 36 - protein, 9 aa, unknown
  MOD_RES (1)...(0): Acyl
PQKSLEEFQ

SEQ ID NO: 37 - protein, 9 aa, unknown
  MOD_RES (1)...(0): Acyl
PQKTLEEFQ

(19) United States
(12) Patent Application Publication
(10) Pub. No.: US 2003/0211589 A1
(43) Pub. Date: Nov. 13, 2003

(54) Short peptides from the b4 and b5 regions kinases which selectively modulate protein activity

(76) Inventor: Shmuel Ben-Sasson, Jerusalem (IL)

Correspondence Address: BROWDY AND NEIMARK, P.L.L.C., 624 NINTH STREET, NW, SUITE 300, WASHINGTON, DC, 20001-5303, US

(21) Appl. No.: 10/149,240

(22) PCT Filed: Dec. 4, 2000
(86) PCT No.: PCT/US00/32852

(51) Int. Cl. A61K 38/53; C12N 9/12; C12P 21/04; C12P 21/02; C12N 5/06; C07K 16/40

(52) U.S. Cl. 435/194; 435/69.1; 435/320.1; 435/325; 424/94.5; 530/388.26; 435/70.21

(57) ABSTRACT

Peptides which are peptide derivatives of the B4-5 region of a protein kinase can modulate the activity of protein kinases. The activity of a protein kinase in a subject can be modulated by administering one or more of these peptides.

### RELATED APPLICATION(S)

This application is a continuation of U.S. Ser. No. 09/458,491, filed Dec. 9, 1999, the entire teachings of which are incorporated herein by reference.

### BACKGROUND OF THE INVENTION

There are a group of proteins that constitute the eukaryotic protein kinase superfamily. Enzymes of this class specifically phosphorylate serine, threonine or tyrosine residues of intracellular proteins. These enzymes are important in mediating signal transduction in multicellular organisms. Many of the protein kinases are part of transmembrane receptors. Others occur as intracellular proteins which take part in signal transduction within the cell, including signal transduction to the nucleus and activation of other proteins. Other protein kinases, such as G protein-coupled receptor kinases, are bound to cell membranes and participate in transmembrane signaling.

As such, phosphorylation of serine, threonine or tyrosine by protein kinases is an important mechanism for regulating intracellular events in response to environmental changes. A wide variety of cellular events are regulated by protein kinases. A few examples include cellular proliferation, cellular differentiation, the ability of cells to enter and/or complete mitosis, cellular transformation by RNA viruses, oncogenesis, immune responses, inflammatory responses and the control of carbohydrate or fat metabolism.

Enhanced protein kinase activity can lead to persistent stimulation by secreted growth factors and other growth inducing factors which, in turn, can lead to proliferative diseases such as cancer, to nonmalignant proliferative diseases such as arteriosclerosis, psoriasis and to inflammatory response such as septic shock. Decreased function can also lead to disease. For example, a decrease in the activity of insulin receptor kinase is a cause of various types of diabetes. Severe reduction of the B cell progenitor kinase leads to human X-linked agammaglobulinemia.

Thus, agents which can modulate (increase or decrease) the activity of protein kinases have great potential for the treatment of a wide variety of diseases and conditions such as cancer, obesity, autoimmune disorders, inflammation and diabetes. Such agents also have utility in deciphering the mode of action of protein kinases and how these proteins regulate cellular functions and activities.

### SUMMARY OF THE INVENTION

It has now been found that short peptides which are derivatives of the b4 to b5 region of a protein kinase (hereinafter designated as B4-5) can significantly affect the activities of cells expressing the protein kinase when incubated with the cells (the “B4-5 region” is defined hereinbelow). For example, the peptide derivatives of the B4-5 region of ActR-IIB stimulate fibroblast cell proliferation (Example 2) while the B4-5 region of ALK3 inhibits neuroblastoma cell proliferation (Example 3). Based on the aforementioned discoveries, novel peptides are disclosed herein which are peptide derivatives of the B4-5 region of protein kinases. Also disclosed are methods of identifying a peptide derivative of a B4-5 region of a protein kinase that modulates the activity of the protein kinase. Methods of modulating the activity of a protein kinase in a subject are also disclosed.

One embodiment of the present invention is a novel peptide which is a peptide derivative of the B4-5 region of a protein kinase. The peptide comprises between about five and about twenty five amino acid residues or amino acid residue analogs of the B4-5 region. The peptide modulates the activity of the protein kinase. The N-terminus and/or C-terminus of the peptide can be substituted or unsubstituted. The peptide can be linear or cyclic.

Another embodiment of the present invention is a method of modulating the activity of a protein kinase in a subject. The method comprises administering a therapeutically effective amount of a peptide that is a derivative of the B4-5 region of the protein kinase, as described above.

Yet another embodiment of the present invention is a method of identifying a peptide which modulates the activity of a protein kinase. The method comprises providing a “test peptide” which has from about five to about twenty five amino acids or amino acid analogs and which is a peptide derivative of the B4-5 region of the protein kinase. The test peptide is incubated with cells having a cellular activity or function under the control of the protein kinase under conditions suitable for assessing the activity of the protein kinase. The activity of the protein kinase is assessed and compared with the activity of the protein kinase in cells of the same cell type grown under the same conditions in the absence of the test peptide. A greater or lesser activity compared with cells grown in the absence of the test peptide indicates that the test peptide modulates the activity of the protein kinase.

The peptides of the present invention can be used in the treatment of a wide variety of diseases caused by overactivity or underactivity of a protein kinase. Examples include, but are not limited to, favourable tissue remodeling such as bone formation, reduced scar formation, enhanced hair growth, induction of differentiation of pancreatic duct cells, inhibition of the growth of adipose tissue, cancer treatment, diseases caused by proliferation of smooth muscle (e.g. restenosis and atherosclerosis), skin disorders, diabetes, obesity, diseases of the central nervous system, inflammatory disorders, autoimmune diseases and other immune disorders, osteoporosis and cardiovascular diseases. The peptides of the present invention also have in vitro utilities, for example, in the generation of antibodies that specifically bind the protein kinase from which the peptide was derived. These antibodies can be used to identify cells expressing the protein kinase and to study the intracellular distribution of the protein kinase. In addition, the peptides of the present invention can be used to identify and quantitate ligands that bind the B4-5 region of the protein kinase from which the peptide was derived.

### BRIEF DESCRIPTION OF THE FIGURES

FIGS. 1A and 1B comprise a table illustrating the amino acid sequences of the B4-5 region of the following protein kinases:

Glycogen synthase kinase and (GSK3) (SEQ ID NO. 1 to 2); Cyclin-dependent kinases 4 and 6 (CDK) (SEQ ID NO. 3 to 4); TGF receptor type II (SEQ ID NO. 5); Activin receptor type II A and B (ACTR) (SEQ ID NO. 6 to 7); Activin receptor-like kinases 1 through 6 (ALK1, 2, 3, 4, 5, 6) (SEQ ID NO. 8 to 11); Bone morphogenetic protein receptor type II (BMPR) (SEQ ID NO. 12); Extra-cellular signal-regulated kinases 1, 2, and 3 (ERK) (SEQ ID NO. 13 to 15); Jun N-terminal kinases 1, 2, and 3 (JNK) (SEQ ID NO. 16 to 17).

FIGS. 2A and 2B comprise a group of sequences illustrating the consensus amino acid sequences of the B4-5 region found among the family of protein kinases. Also shown are examples of conservative substitutions in these amino acid sequences. An “*” indicates an aliphatic, substituted aliphatic, benzylic, substituted benzylic, aromatic or substituted aromatic ester of glutamic acid or aspartic acid.

FIG. 3 is a Table illustrating the sequences of the following peptides:

GSK3aK017B000; GSK3b K018B601 K018B901; CDK4 K050B601 K050B901; CDK6 K089B901; TGFbRU K093B901; ACTRIIA K095B902; ACTRIIB K096B901; ALK1 K048B901; ALK2 K097B901; ALK3 K098B901; ALK4 K099B901; BMPR-11 K116B901 K116B000; ERK1 K113B000; ERK2 K137B000; ERK3 K138B000; JNK1 K109BOOO; JNK2 K136B000; (SEQ ID NO. 18 to 37, respectively).

Peptides are either N-acetylated, N-stearylated, N-hexylated or N-myristylated and C-amidated. FIG. 3 also indicates from which protein kinase each peptide is derived.

FIG. 4 is a graphical representation of the percent change in cellular proliferation of HFL-1 fibroblasts. The treated cells were incubated with different concentrations of an ALK4-derived peptide.

FIGS. 5A and 5B are pictorial depiction of the outgrowth of HNK-1 neural crest cells from neural primordia explants following incubation with an ALK-4 derived peptide. FIG. 5A is a depiction under phase contrast conditions; FIG. 5B is a depiction under HNK-1 stain conditions.

FIGS. 6A and 6B are pictorial depictions of the formation of bone-like tissue at a fracture site on a rabbit ulna (FIG. 6A, control), following treatment with an ALK-4 derived peptide (FIG. 6B).

### DETAILED DESCRIPTION OF THE INVENTION

A protein kinase (hereinafter “PK”) is an intracellular or membrane bound protein which uses the gamma phosphate of ATP or GTP to generate phosphate monoesters on the hydroxyl group of a serine or threonine residue, or on the phenolic group of a tyrosine residue. PKs have homologous “kinase domains” or “catalytic domains” which carry out this phosphorylation. Based on a comparison of a large number of protein kinases, it is now known that the kinase domain of protein kinases can be divided into twelve subdomains. These are regions that are generally uninterrupted by large amino acid insertions and which contain characteristic patterns of conserved residues (Hanks and Hunter, “The Eukaryotic Protein Kinase Superfamily”, in Hardie and Hanks ed.,The Protein Kinase Facts Book, Volume I, Academic Press, Chapter 2, 1995). These subdomains are referred to as Subdomain I through Subdomain XII.

The “B4-5 region” referred to herein is found within the kinase domain of PKs in Subdomain TV and the beginning of Subdomain V. Because of the high degree of homology found in the subdomains of different protein kinases, the amino acid sequences of the domains of different PKs can be aligned. Thus, the B4-5 region of a PK can be defined by reference to the amino acid sequence of a prototypical protein kinase, for example PKA-C, and can be said to correspond to a contiguous sequence of about nine amino acid residues found between about amino acid 106 and 114 of PKA-C. Of course, in some PKs, extra amino acids might be inserted in this region and the size of the B4-5 region can, therefore, include more than 9 amino acids in length.

A second definition of the B4-5 region of a PK, which is complementary to the definition provided in the preceding paragraph, can be made by reference to the three dimensional structure of the kinase domain of PKs. The kinase domain of PKs has been found to contain at least nine alpha helices, referred to as helix A through helix I and nine beta sheets, referred to as b1 through b9 (Tabor et al., Phil. Trans. R. Soc. Lond. B340:315 (1993), Mohammadi et al., Cell 86:577 (1996) and Hubbard et al., Nature 372:746 (1994)). The B4-5 region is a contiguous sequence of about five to twenty five amino acids beginning at the end of the b4 beta sheet and into the b5 beta sheet.

Optionally, the C-terminus or the N-terminus of the peptides of the present invention, or both, can be substituted with a carboxylic acid protecting group or an amine protecting group, respectively. Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the peptide into a cell, for example, by reducing the hydrophilicity and increasing the lipophilicity of the peptide. Examples of N-terminal protecting groups include acyl groups (—CO—R₁) and alkoxy carbonyl or aryloxy carbonyl groups (—CO—O—R₁), wherein R₁ is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, phenyl-CO—, substituted phenyl-CO—, benzyl-CO— and (substituted benzyl)-CO—. Examples of alkoxy carbonyl and aryloxy carbonyl groups include CH₃—O—CO—, (ethyl)-O—CO—, n-propyl-O-CO—, iso-propyl-O—CO—, iso-butyl-O—CO—, sec-butyl-O—CO—, t-butyl-O—CO—, phenyl-O—CO—, substituted phenyl-O—CO— and benzyl-O—CO—, (substituted benzyl)-O—CO—. In order to facilitate the N-acylation, a glycine can be added to the N-terminus of the sequence. The carboxyl group at the C-terminus can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. -In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

A “peptide derivative of the B4-5 region” includes a peptide having the amino acid sequence of the B4-5 region. A “peptide derivative of the B4-5 region” also includes a subsequence of the B4-5 region of the PK. A subsequence of a protein region is a contiguous sequence of from about five to about twenty five amino acids or amino acid residues found within a larger sequence. Thus, a subsequence of the B4-5 region is a contiguous sequence of from about five to about twenty five amino acids or amino acid residues found within the B4-5 region. A subsequence of the B4-5 region can also be referred to as a “fragment” of the B4-5 region.

A “peptide derivative” also includes a peptide having a “modified sequence” in which one or more amino acids in the original sequence or subsequence have been substituted with a naturally occurring amino acid or amino acid analog (also referred to as a “modified amino acid”). In one aspect of the present invention, the peptide derivative has a sequence corresponding to a subsequence of the B4-5 region of a PK, with the proviso that any one amino acid residue in the peptide derivative can differ from the corresponding amino acid residue in the subsequence. For example, if the subsequence is [AA₁]-[AA₂]-[AA₃]-[AA₄]-[AA₅], then the peptide derivative can be [AA₁′]-[AA₂]-[AA₃]-[AA₄]-[AA₅], [AA₁]-[AA₂′]-[AA₃]-[AA₄]-[AA₅], [AA₁]-[AA₂]-[AA₃′]-[AA₄]-[AA₅], [AA₁]-[AA₂]-[AA₃]-[AA₄′]-[AA₅] and [AA₁]-[AA₂]-[AA₃]-[AA₄]-[AA₅′], wherein [AA′] is a naturally occurring or modified amino acid different from [AA]. In another aspect of the present invention, the peptide derivative has a sequence corresponding to a subsequence of the B4-5 region of a PK, with the proviso that any two amino acid residues in the peptide derivative can differ from the corresponding amino acid residue in the subsequence.

An “amino acid residue” is a moiety found within a peptide and is represented by —NH—CHR—CO—, wherein R is the side chain of a naturally occurring amino acid. When referring to a moiety found within a peptide, the terms “amino acid residue” and “amino acid” are used interchangeably in this application. An “amino acid residue analog” includes D or L residues having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. When referring to a moiety found within a peptide, the terms “amino acid residue analog” and “amino acid analog” are used interchangeably in this application. As used herein, aliphatic groups include straight chained, branched or cyclic C1-C8 hydrocarbons that are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include -OH, halogen (—Br, —Cl, —I and —F), —O (aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group), —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(—NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Suitable substitutions for amino acid residues in the sequence of a B4-5 region or a subsequence of a B4-5 region include conservative substitutions which result in peptide derivatives which modulate the activity of a PK. A “conservative substitution” is a substitution in which the substituting amino acid (naturally occurring or modified) has about the same size and electronic properties as the amino acid being substituted. Thus, the substituting amino acid would have the same or a similar functional group in the side chain as the original amino acid.

A “conservative substitution” also refers to utilizing a substituting amino acid that is identical to the amino acid being substituted except that a functional group in the side chain is functionalized with a suitable protecting group. Suitable protecting groups are described in Green and Wuts, “Protecting Groups in Organic Synthesis”, John Wiley and Sons, Chapters 5 and 7, 1991, the teachings of which are incorporated herein by reference. As with N-terminal and C-terminal protecting group, preferred protecting groups are those which facilitate transport of the peptide into a cell, for example, by reducing the hydrophilicity and increasing the lipophilicity of the peptide, and which can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58.557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting, i.e. that there are additional modified amino acids which could be included in each group.

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably, Group I includes leucine, isoleucine, valine and methionine.

Group II includes glycine, alanine, valine, serine, cysteine, threonine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, aspargine, methyl aspartate, ethyl aspartate, benzyl aspartate and methyl glutamate, ethyl glutamate and benzyl glutamate.

Group V includes histidine, lysine, arginine, N-nitroarginine, λ-cycloarginine, g-hydroxyarginine, N-amidinocitruline and 2-amino-4-guanidinobutanoic acid, homologs of lysine, homologs of arginine and ornithine. Preferably, Group V includes histidine, lysine, arginine, and ornithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

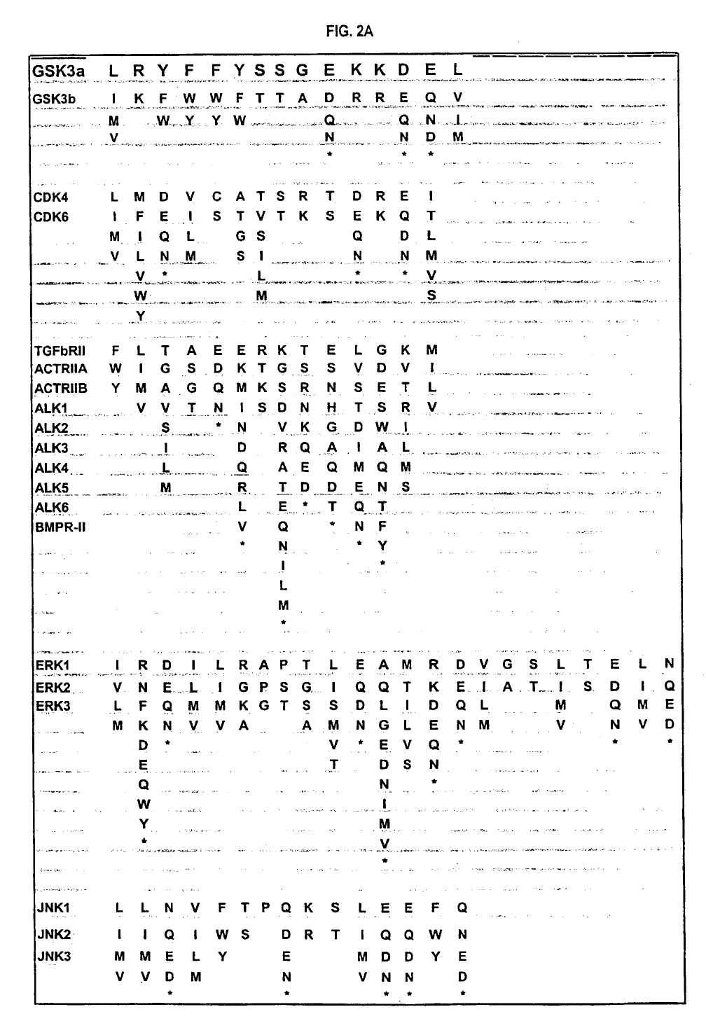

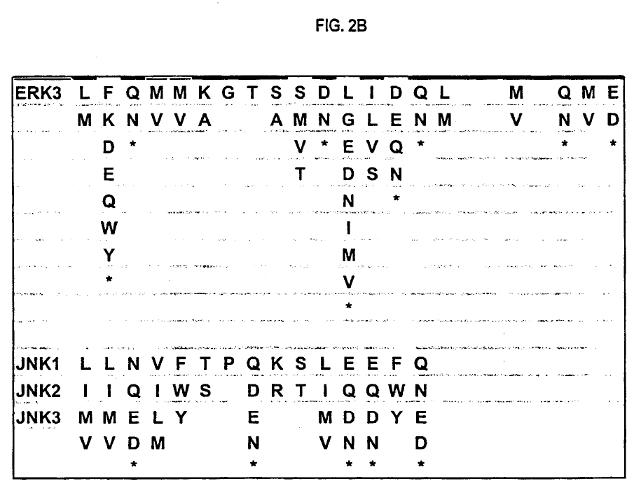

In another aspect, suitable substitutions for amino acid residues in the sequence of a B4-5 region or a subsequence of a B4-5 region include “severe substitutions” which result in peptide derivatives which modulate the activity of a PK. Severe substitutions which result in peptide derivatives that modulate the activity of a PK are much more likely to be possible in positions which are not highly conserved throughout the family of protein kinases than at positions which are highly conserved. FIG. 2 shows the consensus sequences of the five to twenty five amino acids of the B4-5 region of PKs. Because D-amino acids have a hydrogen at a position identical to the glycine hydrogen side-chain, D-amino acids or their analogs can often be substituted for glycine residues.

A “severe substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cycohexylmethyl glycine for alanine, isoleucine for glycine, or —NH—CH[(—CH₂)₅—COOH]—CO— for aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

“Peptidomimetics” can be substituted for amino acid residues in the peptides of this invention. These peptidomimetics replace amino acid residues or act as spacer groups within the peptides. The peptidomimetics often have steric, electronic or configurational properties similar to the replaced amino acid residues but such similarities are not necessarily required. The only restriction on the use of peptidomimetics is that the peptides retain their protein kinase modulating activity. Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110, 5875-5880 (1988)); isosteres of amide bonds (Jones et al., Tetrahedron Lett. 29, 3853-3856 (1988)); LL-3-amino-2-propenidone-6-carboxylic acid (LL-Acp) (Kemp et al., J. Org. Chem. 50, 5834-5838 (1985)). Similar analogs are shown in Kemp et al., Tetrahedron Lett. 29, 5081-5082 (1988) as well as Kemp et al., Tetrahedron Lett. 29, 5057-5060 (1988), Kemp et al., Tetrahedron Lett. 29, 4935-4938 (1988) and Kemp et al., J. Org. Chem.54, 109-115 (1987). Other suitable peptidomimetics are shown in Nagai and Sato, Tetrahedron Lett. 26, 647-650 (1985); Di Maio et al., J. Chem. Soc. Perkin Trans., 1687 (1985); Kahn et al., Tetrahedron Lett. 30, 2317 (1989); Olson et al., J. Am. Chem. Soc. 112, 323-333 (1990); Garvey et al., J. Org. Chem. 56, 436 (1990). Further suitable peptidomimetics include hydroxy-1,2,3,4-tetrahydroisoquinoline-3-carboxylate (Miyake et al., J. Takeda Res. Labs 43, 53-76 (1989)); 1,2,3,4-tetrahydroisoquinoline-3-carboxylate (Kazmierski et al, J. Am. Chem. Soc. 133, 2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

The amino acid residues of the peptides can be modified by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang Chem. Int. Ed. English 26, 294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

Examples of PKs whose activity can be modulated by peptide and peptide derivatives, as described herein, include, but are not limited to, PKs belonging to the following PK families: Mitogen-activated protein kinases (MAP kinases), Cyclin-Dependent kinases (CDKs), Activin receptors (ACTRs) and activin-like kinase receptors (ALKs), TGFb receptors, glycogen synthase kinase-3 (GSK3). Suitable members from the MAP kinase family include, but are not limited to, ERK1, ERK2, ERK3, JNK1, JNK2, and JNK3. Suitable members from the CDK family include, but are not limited to, CDK4, and CDK6. Suitable members from the ACTR family include, but are not limited to, ACTRIIA, and ACTIIB. Suitable members from the ALK family include, but are not limited to, ALK1, ALK2, ALK3, ALK4, ALK5, and ALK6. Suitable members from the TGFbR family include, but are not limited to, TGFbRII and TGFbRI. Suitable members from the GSK3 family include, but are not limited to, GSK3a, and GSK3b. Other suitable PKs include, but are not limited to, Bone morphogenetic protein receptor II (BMPRII).

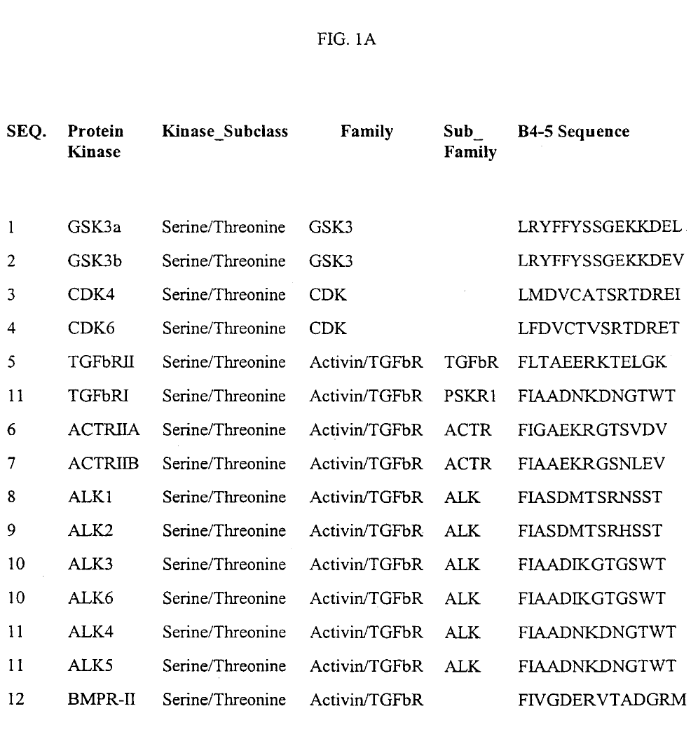

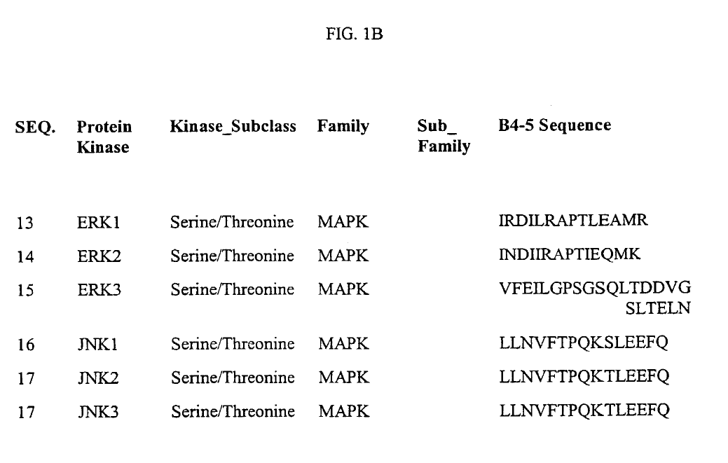

As shown in FIG. 1, the sequences of suitable peptide members of the B4-5 region of PKs from different families include, but are not limited to:

Glycogen synthase kinase and (GSK3) (SEQ ID NO. 1 to 2); Cyclin-dependent kinases 4 and 6 (CDK) (SEQ ID NO. 3 to 4); TGF receptor type II (SEQ ID NO. 5); Activin receptor type II A and B (ACTR) (SEQ ID NO. 6 to 7); Activin receptor-like kinases 1 through 6 (ALK1, 2, 3, 4, 5, 6) (SEQ ID NO. 8 to 11); Bone morphogenetic protein receptor type II (BMPR) (SEQ ID NO. 12); Extra-cellular signal-regulated kinases 1, 2, and 3 (ERK) (SEQ ID NO. 13 to 15); Jun N-terminal kinases 1, 2, and 3 (JNK) (SEQ ID NO. 16 to 17).

The amino acid at the N-terminus of the B4-5 region is at position I and can be referred to as “[AA]₁”. The next amino acid in the sequence, referred to as “[AA]₂”, is at position 2 and is followed by amino acids [AA]₃ through [AA]ₘ, which are at positions 3 to m, where in is the position number of the amino acid at the C-terminus of the B4-5 region. Likewise, (m⁻¹²) is the position number of the amino acid twelve amino acid residues before the C-terminus of the B4-5 region. Thus, a peptide 20-mer with an amino acid sequence [AA₁] through [AA₂₀] includes the first twenty amino acids in the B4-5 region. A peptide derivative of the B4-5 region with an amino acid sequence [AA₅] through [AA₁₆] includes the fifth amino acid through the sixteenth amino acid in the B4-5 region, and a peptide derivative of the B4-5 region with an amino acid sequence [AA]₍ₘ₋₂) through [AA]ₘ includes the last twelve amino acids in the B4-5 region. In this invention, in can have a value between 10 and 25.

The present invention includes peptides having amino acid sequences corresponding to the sequence found in the B4-5 region of PKs, subsequences thereof and modified subsequences thereof. Examples of suitable subsequences include, but are not limited to, sequences corresponding to [AA₁]through [AAₘ], [AA] ₁ through [AA]₁₂[AA]₅ through [AA]₁₆, [AA]₉ through [AA]₂₀, [AA]₍ₘ₋₂₎ through [AA]ₘ, [AA]₍ₘ₋₁₂₎ through [AA]₍ₘ₋₂₎ and [AA]₍ₘ₋₂₀₎ through [AA]₍ₘ₋₈₎ of the B4-5 region of a PK, and subsequences thereof. The above designated sequences are preferred.

The present invention includes peptides having amino acid sequences corresponding to a modified sequence or subsequence of the B4-5 region of PKs and which modulate the activity of PKs including: GSK3a; GSK3b; CDK4; CDK6; TGFbRII; TGFbRI; ACTRIIA; ACTRIIB; ALK1; ALK2; ALK3; ALK4; ALK5; ALK6; BMPR-II; ERK1; ERK2; ERK3; JNK1; JNK2; JNK3.

In one aspect, one, two or more of the amino acids in the sequence or subsequence are modified with conservative substitutions; the substitutions can be in consensus positions, in non-consensus positions or in both. In another aspect, one, two or more of the amino acids in the sequence or subsequence are modified with severe substitutions; the substitutions are preferably in non-consensus positions. FIG. 2 provides examples of conservative amino acid substitutions for the B4-5 region of:

Glycogen synthase kinase and (GSK3) (SEQ ID NO. 1 to 2); Cyclin-dependent kinases 4 and 6 (CDK) (SEQ ID NO. 3 to 4); TGF receptor type II (SEQ ID NO. 5); Activin receptor type II A and B (ACTR) (SEQ ID NO. 6 to 7); Activin receptor-like kinases 1 through 6 (ALK1, 2, 3, 4, 5, 6) (SEQ ID NO. 8 to 11); Bone morphogenetic protein receptor type II (BMPR) (SEQ ID NO. 12); Extra-cellular signal-regulated kinases 1, 2, and 3 (ERK) (SEQ ID NO. 13 to 15); Jun N-terminal kinases 1, 2, and 3 (JNK) (SEQ ID NO. 16 to 17).

The conservative substitutions can occur by exchanging amino acids with aligned B4-5 region sequences, as shown in FIG. 2, as well as by substituting the listed amino acids that are not associated with a known B4-5 region sequence.

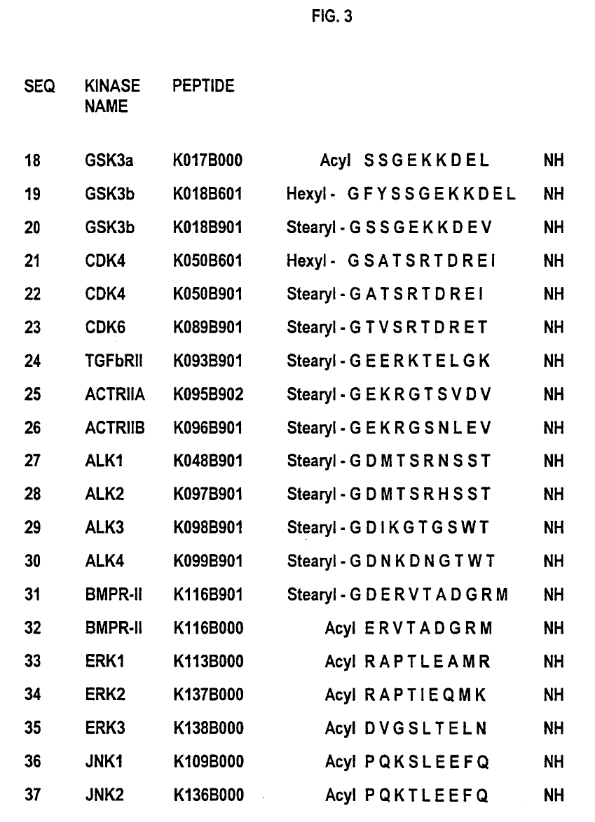

Specific examples of peptide derivatives of the present invention include peptides: GSK3a K017B000; GSK3b K08B601 K018B901; CDK4 K050B601 K050B901; CDK6 K089B901; TGFbRII K093B901; ACTRIIA K095B902; ACTRIIB K096B901; ALK1 K048B901; ALK2 K097B901; ALK3 K098B901; ALK4 K099B901; BMPR-II K116B901 K116B000; ERK1 K113B000; ERK2 K137B000; ERK3 K138B000; JNK1 K109B000; JNK2 K136B000; (SEQ ID NO. 18 to 37, respectively), as specified in FIG. 3.

The N-terminus and/or C-terminus of these peptides can be modified, as described above and as shown in FIG. 3. The N-terminal of these peptides is acetylated, stearylated, hexylated or myristylated and the C-terminal is amidated. Other protecting groups for amides and carboxylic acids can be used, as described above. Optionally, one or both protecting groups can be omitted. The peptides may be linear or cyclic.

Also included are peptides having the sequence of:

GSK3a K017B000; GSK3b K018B601 K018B901; CDK4 K050B601 K050B901; CDK6 K089B901; TGFbRH K093B901; ACTRIIA K095B902; ACTRIIB K096B901; ALK1 K048B901; ALK2 K097B901; ALK3 K098B901; ALK4 K099B901; BMPR-II K116B901 K116B000; ERK K113B000; ERK2 K137B000; ERK3 K138B000; JNK1 K109BOOO; JNK2 K136B000; (SEQ ID NO. 18 to 37, respectively), as specified in FIG. 3, with the proviso that any one or two of the amino residues in the peptide can vary, being replaced by any naturally occurring amino acid or analog thereof.

The present invention also includes cyclic peptides having amino acid sequences corresponding to a modified sequence or subsequence of the B4-5 region of PKs. These cyclic peptides modulate the activity of PKs.

A “cyclic peptide” refers, for example, to a peptide or peptide derivative in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the peptide, preferably the side chain of the C-terminal amino acid. For example, an amide can be formed between the nitrogen atom at the N-terminus and the carbonyl carbon in the side chain of an aspartic acid or a glutamic acid. Alternatively, the peptide or peptide derivative can be cyclized by forming a covalent bond between the carbonyl at the C-terminus of the compound and the side chain of a suitable amino acid in the peptide, preferably the chain of the N-terminal amino acid. For example, an amide can be formed between the carbonyl carbon at the C-terminus and the amino nitrogen atom in the side chain of a lysine or an ornithine. Additionally, the peptide or peptide derivative can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the peptide, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in side chain of, for example, a lysine or an ornithine.

In addition, a peptide or peptide derivative can be cyclized with a linking group between the two termini, between one terminus and the side chain of an amino acid in the peptide or peptide derivative, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

Suitable substitutions in the original amino acid sequence or subsequence are those which result in a peptide derivative, as defined above, which modulates the activity of a PK. The activity of a PK is “modulated” when the activity of the PK is increased or decreased. An increase or decrease in the activity of a PK can be detected by assessing in vitro the extent of phosphorylation of a protein substrate of the PK being tested or by a corresponding modulation, increase or decrease, in a cellular activity or function which is under the control of the PK. Examples of these cellular functions include cell proliferation, cell differentiation, cell morphology, cell survival or apoptosis, cell response to external stimuli, gene expression, lipid metabolism, glycogen or glucose metabolism and mitosis.

It can be readily determined whether a peptide or peptide derivative modulates the activity of a PK by incubating the peptide or peptide derivative with cells which have one or more cellular activities controlled by a PK. The cells are incubated with the peptide or peptide derivative to produce a test mixture under conditions suitable for assessing the activity of the protein kinase. The activity of the PK is assessed and compared with a suitable control, e.g., the activity of the same cells incubated under the same conditions in the absence of the peptide or peptide derivative. A greater or lesser activity of the PK in the test mixture compared with the control indicates that the test peptide or peptide derivative modulates the activity of the PK.

Suitable cells for the assay include normal cells which express a membrane bound or intracellular PK, cells which have been genetically engineered to express a PK, malignant cells expressing a PK or immortalized cells which express a PK.

Conditions suitable for assessing PK activity include conditions suitable for assessing a cellular activity or function under control of the PK. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors).

In another aspect, the activity of certain PK (e.g., Atk/PKB, Dudek et al., Science 275:661 (1997)) can be evaluated by growing the cells under serum deprivation conditions. Cells are typically grown in culture in the presence of a serum such as bovine serum, horse serum or fetal calf serum. Many cells, for example, nerve cells such as PC-12 cells, generally do not survive with insufficient serum. The use of insufficient serum to culture cells is referred to as “serum deprivation conditions” and includes, for example, from 0% to about 4% serum. PK activity is determined by the extent to which a peptide or peptide derivative can protect cells, e.g., neuronal cells, from the consequences of serum deprivation. Specific conditions are provided in Dudek et al., and in Example 4 of co-pending and concurrently filed application entitled “SHORT PEPTIDES WHICH SELECTIVELY MODULATE INTRACELLULAR SIGNALING” (filed on May 21, 1997, U.S. application Ser. No. 08/861,153), the teachings of which are incorporated herein by reference.

Generally, the activity of the PK in the test mixture is assessed by making a quantitative measure of the cellular activity which the PK controls. The cellular activity can be, for example, cell proliferation. Examples of cells in which proliferation is controlled by a PK include endothelial cells such as bovine aortic cells, mouse MSI cells or mouse SVR cells (see Arbiser et al., Proc. Natl. Acad. Sci. USA 94:861 (1997)), vascular smooth muscle cells, fibroblasts of various tissue origin, and malignant cells of various tissues such as breast cancer, lung cancer, colon cancer, prostate cancer or melanoma. PK activity is assessed by measuring cellular proliferation, for example, by comparing the number of cells present after a given period of time with the number of cells originally present. One example of PKs having to do with cellular proliferation are the receptors of the TGFb super-family.

Specific examples of conditions suitable for determining the activity of PKs by assessing cell proliferation are provided in Example 2.

If cells are being used in which the PK controls cell differentiation (e.g., preadipocytes such as 3T3-L1 expressing PKs Akt/PKB, GSK3 and protein kinase A—see Kohn et al., J. Biol. Chem. 271:31372 (1996)), activity is assessed by measuring the degree of differentiation. Activity can be assessed by changes in the metabolic activity of cells such as primary adipocytes, hepatocytes and fibroblasts by measuring changes in glucose uptake, lipogenesis, or glycogen metabolism (see, for example, Weise et al., J. Biol. Chem. 270:3442 (1995)). Activity can also be assessed by the extent to which gene expression, cell morphology or cellular phenotype is altered (e.g., the degree to which cell shape is altered or the degree to which the cells assume a spindle-like structure). One example of a change in cellular morphology is reported in the co-pending and concurrently filed application entitled “SHORT PEPTIDES WHICH SELECTIVELY MODULATE INTRACELLULAR SIGNALING” (filed on May 21, 1997, U.S. application Ser. No. 08/861,153), which discloses that certain peptide derivatives of the HJ loop of protein tyrosine kinases can cause vascular smooth muscle cells to become elongated and assume a spindle-like shape.

It is to be understood that the assay described hereinabove for determining whether a peptide or peptide derivative modulates a cellular activity or function under the control of a PK can be performed with cells other than those specifically described herein. PKs not yet discovered or PKs whose function is not yet known can also be used in this assay, once it has been determined which cellular functions or activities they control. These PKs are also within the scope of the present invention.

The present invention is also directed to a method of modulating the activity of a protein kinase in a subject. A “subject” is preferably a human, but can also be animals in need of treatment, e.g., veterinary animals (e.g., dogs, cats, and the like), farm animals (e.g., cows, pigs, horses, chickens and the like) and laboratory animals (e.g., rats, mice, guinea pigs and the like).

The activity of a PK in a subject can be modulated for the purpose of treating diseases that are caused by over activity or under activity of PKs. For example, MAP kinases (Seger and Krebs, FASEB J. 9:726 (1995)) and cyclin dependent protein kinases (“Molecular Biology of the Cell,” Alberts, Bray, Lewis, Raff, Roberts and Watson, eds. Chapter 5, (Garland Publishing, Inc.), (1994)), are central components of the cell-division cycle control system in eukaryotic cells. Peptides and peptide derivatives of the present invention which modulate the activity of these enzymes can be used to treat cancer in a subject when administered to the subject in a therapeutically effective amount.

GSK3 family members are involved in the control of glycogen metabolism. Peptide and peptide derivatives of the present invention which modulate the activity of glycogen synthase kinase 3 can be used to treat Type II diabetes and hemorrhagic shock in a subject when administered to the subject in a therapeutically effective amount.

Inhibition of GSK3 might also increase the intracellular activity of the insulin receptor and thereby enhance glucose uptake and other related metabolic activities. Thus, agents which modulate the activity of GSK3 can be useful in the treatment of Type I and Type II diabetes.

Another family of transmembrane protein kinases is composed of members of the TGF/Activin/BMP receptors which transduce signals of the corresponding cytokines. The TGF/Activin/BMP cytokines participate in various processes of tissue remodelling, including the induction of bone formation, hair growth, adipose tissue proliferation and differentiation of pancreatic islet cells. Therefore, modulation of the activity of these receptor kinases can assist tissue repair, inhibit tissue fibrosis and fat tissue growth, assist in hair growth, induce differentiation of pancreatic β-cells and enhance bone formation.

Based on methods disclosed herein, peptides and peptide derivatives can be designed to modulate the activity of PKs whose B4-5 region has been sequenced or will be sequenced in the future and whose cellular function is known. As a consequence, peptides and peptide derivatives can be designed to affect (increase or decrease) those cellular functions. It is possible that future research will reveal that certain disease conditions, whose underlying causes are presently unknown, are brought about by the overactivity or underactivity of cellular functions controlled by these PKs. These diseases can be treated by administering peptides which are peptide derivatives of the B4-5 region of the overactive or underactive PK. Suitable peptides and peptide derivatives can be identified by methods disclosed herein. These methods of treatment, peptides and peptide derivatives are encompassed within the scope of the present invention.

A “therapeutically effective amount” is the quantity of compound which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment. With respect to cancer, an “improved clinical outcome” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

With respect to diabetes, an improved clinical outcome refers to a longer life expectancy, a reduction in the complications of the disease (e.g., neuropathy, retinopathy, nephropathy and degeneration of blood vessels) and an improved quality of life, as described above.

With respect to obesity, an improved clinical outcome refers to increased weight reduction per caloric intake or a reduction in food intake. It also refers to a decrease in the complications which are a consequence of obesity, for example heart disease such as arteriosclerosis and high blood pressure.

The amount of peptide or peptide derivative administered to the individual will depend on the type and severity of the disease and on the characteristics of the individual, such as general health, age, sex, body weight and tolerance to drugs. The skilled artisan will be able to determine appropriate dosages depending on these and other factors. Typically, a therapeutically effective amount of the peptide or peptide derivative can range from about 1 mg per day to about 1000 mg per day for an adult. Preferably, the dosage ranges from about 1 mg per day to about 100 mg per day.

The peptide and peptide derivatives of the present invention are preferably administered parenterally. Parenteral administration can include, for example, systemic administration, such as by intramuscular, intravenous, subcutaneous, or intraperitoneal injection. Peptides or peptide derivatives which resist proteolysis can be administered orally, for example, in capsules, suspensions or tablets. The peptide or peptide derivative can also be administered by inhalation or insufflation or via a nasal spray.

The peptide or peptide derivative can be administered to the individual in conjunction with an acceptable pharmaceutical carrier as part of a pharmaceutical composition for treating the diseases discussed above. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986).

The peptide and peptide derivatives of the present invention have many utilities other than as a therapeutic agent. Some of these uses are discussed in the following paragraphs.

The B4-5 region peptides of the present invention are derived from an array which is linear in the native protein. These peptides can be useful in the preparation of specific antibodies against PKs. Moreover, since the B4-5 region sequence is unique to each sub-family of PK, anti-B4-5 region antibodies can be specifically used to isolate distinct sub-families of PK.

Suitable antibodies can be raised against a B4-5 region peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Antibodies, including monoclonal antibodies, against B4-5 region peptides have a variety of uses. For example, those against or reactive with the protein from which the B4-5 peptides was derived, and preferably which bind specifically to said protein, can be used to identify and/or sort cells exhibiting that protein on the cell surface (e.g., by means of fluorescence activated cell sorting or histological analyses). Monoclonal antibodies specific for the protein can also be used to detect and/or quantitate the protein expressed on the surface of a cell or present in a sample (e.g., in an ELISA). Alternatively, the antibodies can be used to determine if an intracellular PK is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-B4-5 region antibody specific for the PK. The lysate is then analyzed, for example, by Western blotting or immunoprecipitation for complexes between PK and antibody. Some PKs become membrane-bound or cytoskeleton-associated following stimulation. Anti-B4-5 region antibodies can be utilized for the study of the intracellular distribution (compartnentalization) of various subfamilies of PKs under various physiological conditions via the application of conventional immunocytochemistry such as immunofluorescence, immunoperoxidase technique and immunoelectron microscopy, in conjunction with the specific anti-B4-5 region antibody. Antibodies reactive with the B4-5 region are also useful to detect and/or quantitate the PK or B4-5 peptide in a sample, or to purify the PK from which the B4-5 region was derived (e.g., by immunoaffinity purification).

The B4-5 region within PKs plays a key role in regulating the activity of PKs, as is evidenced by the fact that the peptides and peptide derivatives of the present invention have such a dramatic effect on the activity of PKs. The B4-5 region peptides of the present invention can also be used to identify ligands which interact with the B4-5 regions of specific PKs and which modulate the activity PKs. For example, an affinity column can be prepared to which a specific B4-5 region peptide is covalently attached, directly or via a linker. This column, in turn, can be utilized for the isolation and identification of specific ligands which bind the B4-5 region peptide and which will also likely bind the PK from which the B4-5 region peptide was derived. The ligand can then be eluted from the column, characterized and tested for its ability to modulate PK function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these references are incorporated herein by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

The invention is illustrated by the following examples which are not intended to be limiting in any way.

### EXAMPLE 1

### Preparation of B4-5 Peptides

The novel compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J Org. Chem., 37: 3404 (1972); Gauspohl, H., et al., Synthesis, 5: 315 (1992)), the teachings of which are incorporated herein by reference.

Rink Amide Resin [4(2′, 4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzotriazolyl-1-yl)-1,1,3,3-tetramethyluronium) dissolved in HOBt (1-hydroxybenzotriazole) and DMF (dimethylformamide). The amino acid was dissolved in this solution with additional NMP. DIEA (diisopropylethylamine) was added to initiate activation. Alternatively, the activation method of DCC (dicyclohexylcarbodiimide) and HOBt was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisole; 0.5 ml D.I. H₂O; 10 ml TFA.

### EXAMPLE 2

### A B4-5 Peptide Derivative of ALK-4 Modulates Fibroblast Cell Proliferation

Human Fetal lung fibroblasts (HFL-1) were obtained from the American Type Culture Collection (Manassas, Va.).

Culture medium was prepared from F-12K (Gibco), penicillin/streptomycin/glutamine (penicillin—100U/ml; streptomycin—100U/ml; glutamine—2 mM), 0.5% fetal calf serum and 50 mg/ml ascorbic acid. A suspension of HFL-1 cells at 2.22×10⁵ cell/ml was prepared in the above described culture medium.

A series of B4-5 peptide stock solution was prepared by diluting a 10 nM solution of the B4-5 peptide in 100% DMSO, with phosphate buffered saline (PBS) containing 0.1% BSA. The concentration of B4-5 peptide in each stock solution was adjusted to nine times the desired concentration of the B4-5 peptide in the assay mixture. Twenty μl of peptide stock solution were placed at the bottom of each well in a 96-well flat bottom tissue culture plate, six replications for each concentration. BSA solution containing 1% DMSO with no added peptide served as a control. 180 μl of the HFL-1 cell suspension were added to each well (40,000 cells per well). The cells were incubated with the peptide (final concentrations of 0-10μM), for 48 hours at 37° C. in a 5% CO₂ humidified incubator. At the end of the incubation the cells were fixed with buffered formaline (200 μl/well) for 1 h at room temperature. The wells were then washed with 0.1M, pH 8.5 borate buffer (200 μl/well). The fixed cells were stained with freshly filtered 1% methylene blue solution (50 μl/well) for 15 min at room temperature. Excess dye was washed with tap water. Cell-bound dye was eluted with 200 μl of 0.1M HCl per well. The O.D. was read at 595 nm to determine the number of cells per well. The procedure for counting cells is described in greater detail in Oliver et al., J. of Cell Sci., 92:513 (1989), the teaching of which are incorporated herein by reference.

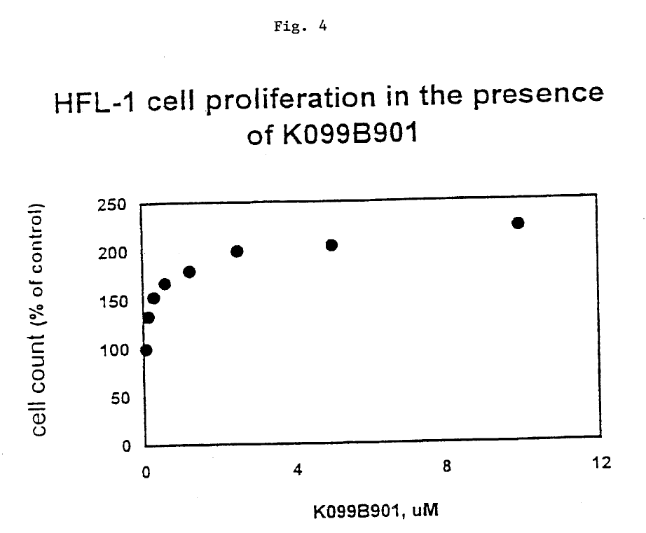

The results for B4-5 peptide K099B901 are shown in FIG. 4, wherein it is evident that this B4-5 peptide stimulates cell proliferation (up to and exceeding 2×) of the HFL-1 cells.

### EXAMPLE 3

### Enhancement of Emigration of Neural Crest Cells From Neural Primordia by B4-5 Peptide

The procedure for explants of neural primordia is described in greater detail in Sela-Donenfeld and Kalcheim (Development, 1999, in press), the teaching of which are incorporated herein by reference.

The trunk region of 16 somite-old quail embryos was separately sectioned at the level of the segmental plate plus the last 2 epithelial somite pairs. Neural primordia consisting of the neural tube and premigratory neural crest cells were isolated from adjacent tissues with 25% pancreatin in PBS, transferred to PBS supplemented with 5% newborn calf serum to stop enzymatic activity and washed in serum-free culture medium prior to explantation. The neural primordia were then explanted onto multi-well chamber slides that were pre-coated with fibronectin (50 μg/ml) for 1 hour. The neural primordia were cultured in 50 μl serum-free SFRI medium (Berganton, France) in the absence or presence of B4-5 peptide—K099B901, in a final concentration of 5 μM and incubated in a humid chamber for 24 hours. At the end of the incubation, the primordia were gently washed with PBS and fixed with Bouin's fluid, washed 3 times with PBS and immunostained with 50 μl of monoclonal antibodies against HNK-1 for 1 h at room temperature. Excess HNK-1 antibodies were washed 3 times with PBS. The samples were further incubated with 50 μl of a secondary fluorescent antibody, GAM-FITC for 1 h at room temperature. Excess antibodies were washed 3 times with PBS. The slides were dried and covered.

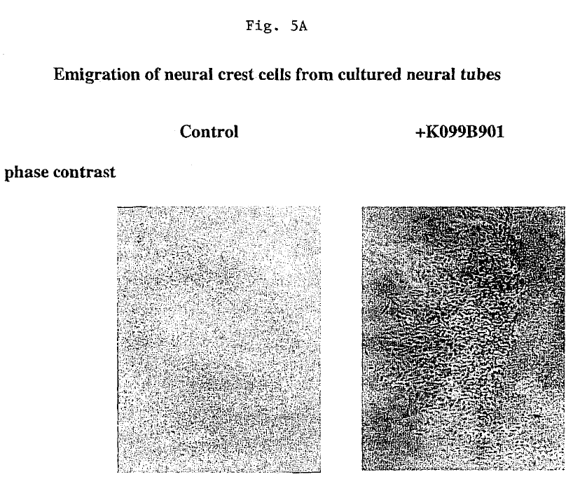

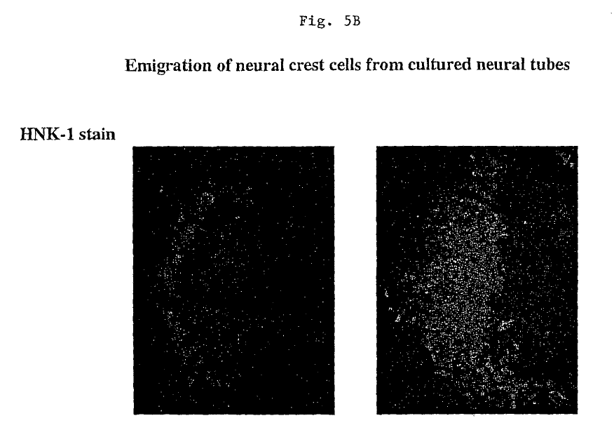

The results for B4-5 peptide K099B901 are shown in FIG. 5.

As can clearly be seen in FIGS. 5A and B, the addition of K099B901 to neural primordia explants caused a remarkable outgrowth of HNK-1—positive neural crest cells from the explant as compared to control.

### EXAMPLE 4

### Enhancement of Bone Remodeling by a B4-5 Peptide

The rabbit ulnar osteotomy models described by Bouxsein et al., at the 45^{th} Annual Meeting, Orthopedic Research Society, Anaheim, Calif. (1999), were employed to assess the ability of the B4-5 peptide K099B901 (ALK4-derived peptide) to accelerate bone remodeling.

Bilateral mid-ulnar osteotomies were made in the front limbs of male New Zealand White rabbits using an oscillating saw. The preparation of the peptide was as follows: 40 mg of peptide were dissolved in 200 μl of DMSO (Sigma) and 800 μl of double distilled water. The solution was mixed with 100 mg of methyl cellulose (Sigma) and 100 mg of carboxymethyl cellulose (Serva).

The K099B901 peptide mixture (0.3 cm³) was locally applied to the fractured area. An untreated osteotomy served as a control. Radiographs of the ulna were taking weekly for 4 weeks. The rabbits were then sacrificed and the fractured areas were taken for bone histology. The bone tissue was fixated in 4% formaldehide and then soaked in 10% formic acid for decalcification. The decalcified tissue was imbedded in paraffin wax, serially sectioned at 5μ and stained with indigo-carmine.

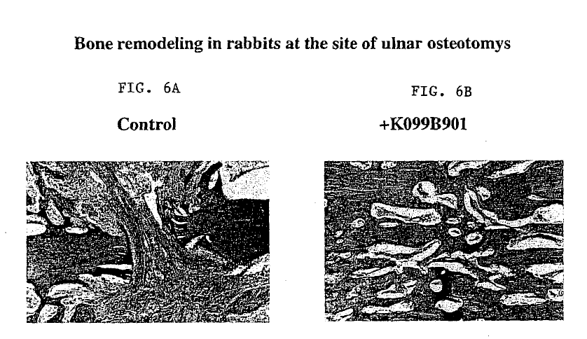

The results for B4-5 peptide K099B901 are shown in FIG. 6.

As can clearly be seen in FIG. 6, while in the control animal a fibrotic connective tissue was formed at the fracture site, in the K099B901-treated animal, a bone like bone-tissue was formed leading to a genuine regeneration.

While this invention has been particularly shown and described with references to preferred embodiments thereof, it will be understood by those skilled in the art that various changes in form and details may be made therein without departing from the scope of the invention encompassed by the appended claims.

What is claimed is:

1. A peptide comprising a peptide derivative of the B4-5 region of a protein kinase, wherein:
a) said peptide has between about five and about twenty-five amino acids or amino acid analogs; and
b) said peptide modulates cellular activity of the protein kinase.

2. The peptide of claim 1 wherein the peptide is cyclic.

3. The peptide of claim 1 wherein the peptide is linear.

4. The peptide of claim 3 wherein the N-terminus and the C-terminus of the peptide are unsubstituted.

5. The peptide of claim 3 wherein at least one of the N-terminus or the C-terminus is substituted.

6. The peptide of claim 5 wherein the N-terminus is amidated and the C-terminus is acylated.

7. The peptide of claim 3 wherein the peptide derivative has an amino acid sequence corresponding to any subsequence of the amino acid sequence of said B4-5 region of said protein kinase, with the proviso that any one amino acid in the sequence of the peptide derivative can vary, being any amino acid or analog thereof.

8. The peptide of claim 3 wherein the protein kinase is member of a protein kinase family selected from the group of families consisting of mitogen-activated protein kinases, cyclin dependent kinases, glycogen synthase kinases, bone morphogenetic protein receptor kinases, TGFb receptor kinases, activin receptor kinases, activin receptor-like kinases, extra-cellular signal-regulated kinases and Jun N-terminal kinases.

9. The peptide of claim 8 wherein the protein kinase is a glycogen synthase kinase selected from the group consisting of GSK3a and GSK3b.

10. The peptide of claim 8 wherein the protein kinase is a cyclin dependent kinase selected from the group consisting of CDK4 and CDK6.

11. The peptide of claim 8 wherein the protein kinase is bone-morphogenetic protein receptor kinase BMPR-II.

12. The peptide of claim 8 wherein the protein kinase is an activin receptor kinase selected from the group consisting of ACTRIIA and ACTRIIB.

13. The peptide of claim 8 wherein the protein kinase is TGF receptor kinase TGFbRII.

14. The peptide of claim 8 wherein the protein kinase is an activin receptor-like kinase selected from the group consisting of TGFbRI, ALK1, ALK2, ALK3, ALK4, ALK5 and ALK6.

15. The peptide of claim 8 wherein the protein kinase is an extra-cellular signal-regulated kinase selected from the group consisting of ERK1, ERK2 and ERK3.

16. The peptide of claim 8 wherein the protein kinase is a Jun N-terminal kinase selected from the group consisting of JNK1, JNK2 and JNK3.

17. The peptide of claim 3 wherein the peptide derivative has an amino acid sequence corresponding to any subsequence of the amino acid sequence of said B4-5 region.

18. The peptide of claim 3 wherein the peptide has the sequence of GSK3a K017B000; GSK3b K018B601 K018B901; CDK4 K050B601 K050B901; CDK6 K089B901; TGFbRII K093B901; ACTRIIA K095B902; ACTRIIB K096B901; ALK1 K048B901; ALK2 K097B901; ALK3 K098B901; ALK4 K099B901; BMPR-II K116B901 K116B000; ERK1 K113B000; ERK2 K137B000; ERK3 K138B000; JNK1 K109B000; JNK2 K136B000; (SEQ ID NO. 18 to 37, respectively,) as specified in FIG. 3.

19. A peptide having the sequence of GSK3a K017B000; GSK3b K018B601 K018B901; CDK4 K050B601 K050B901; CDK6 K089B901; TGFbRII K093B901; ACTRIIA K095B902; ACTRIIB K096B901; ALK1 K04SB901; ALK2 K097B901; ALK3 K098B901; ALK4 K099B901; BMPR-11 K116B901 K116B000; ERK1 K113B000; ERK2 K137B000; ERK3 K138B000; JNK1 K109B000; TNK2 K136B000: (SEQ ID NO. 18 to 37, respectively), as specified in FIG. 3, with the proviso that any one amino acid residue in the peptide can vary, being any naturally occurring amino acid or analog thereof.

20. A peptide comprising a sequence of amino acids AA₁ through AA₁₅ or a subsequence thereof comprising at least five amino acids, wherein:
AA₁ is selected from the group consisting of leucine, methionine, isoleucine and valine;
AA₂ is selected from the group consisting of arginine and lysine;
AA₃ is selected from the group consisting of tyrosine, phenylalanine and tryptophan;
AA₄ is selected from the group consisting of phenylalanine, tryptophan and tyrosine;
AA₅ is selected from the group consisting of phenylalanine, tryptophan and tyrosine;
AA₆ is selected from the group consisting of tyrosine, phenylalanine and tryptophan;
AA₇ is selected from the group consisting of serine and threonine;
AA₈ is selected from the group consisting of serine and threonine;
AA₉ is selected from the group consisting of glycine and alanine;
AA₁₀ is selected from the group consisting of glutamic acid, aspartic acid, glutamine, asparagine and an aliphatic, substituted aliphatic, benzyl, substituted benzyl, aromatic or substituted aromatic ester of glutamic acid or aspartic acid;
AA₁₁ is selected from the group consisting of lysine and arginine;
AA₁₂ is selected from the group consisting of lysine and arginine;
AA₁₃ is selected from the group consisting of aspartic acid, glutamic acid, glutamine, asparagine and an aliphatic, substituted aliphatic, benzyl, substituted benzyl, aromatic or substituted aromatic ester of glutamic acid or aspartic acid;
AA₁₄ is selected from the group consisting of glutamic acid, glutamine, asparagine, aspartic acid and an aliphatic, substituted aliphatic, benzyl, substituted benzyl, aromatic or substituted aromatic ester of glutamic acid or aspartic acid; and
AA₁₅ is selected from the group consisting of leucine, valine, isoleucine and methionine.

21. The peptide of claim 20 wherein the sequence AA₁ through AA₁₅ or a subsequence thereof corresponds to a sequence of the B4-5 region of a glycogen synthase kinase selected from the group consisting of SEQ ID NO: 1 and SEQ ID NO: 2 or a subsequence thereof, with the proviso that any two amino acids in the sequence AA₁ through AA₁₅ or the subsequence thereof can vary.

22. The peptide of claim 20 wherein the sequence AA₁ through AA₁₅ or a subsequence thereof corresponds to the sequence of the B4-5 region of a glycogen synthase kinase selected from the group consisting of SEQ ID NO: 1 and SEQ ID NO: 2 or a subsequence thereof, with the proviso that any one amino acid in the sequence AA₁ through AA₁₅ or the subsequence thereof can vary.

23. A peptide comprising a sequence of amino acids AA₁ through AA₁₄ or a subsequence thereof comprising at least five amino acids, wherein:
AA₁ is selected from the group consisting of leucine, isoleucine, methionine and valine;
AA₂ is selected from the group consisting of methionine, phenylalanine, isoleucine, leucine, valine, tryptophan, and tyrosine;
AA₃ is selected from the group consisting of aspartic acid, glutamic acid, glutamine, asparagine and an aliphatic, substituted aliphatic, benzyl, substituted benzyl, aromatic or substituted aromatic ester of glutamic acid or aspartic acid;
AA₄ is selected from the group consisting of leucine, valine, isoleucine and methionine;
AA₅ is selected from the group consisting of cysteine and serine;
AA₆ is selected from the group consisting of alanine, threonine, glycine and serine;
AA₇ is selected from the group consisting of threonine, valine, serine, isoleucine, leucine and methionine;
AA₈ is selected from the group consisting of serine and threonine;
AA₉ is selected from the group consisting of arginine and lycine;
AA₁₀ is selected from the group consisting of threonine and serine;
AA₁₁ is selected from the group consisting of aspartic acid, glutamic acid, glutamine, asparagine and an aliphatic, substituted aliphatic, benzyl, substituted benzyl, aromatic or substituted aromatic ester of glutamic acid or aspartic acid;
AA₁₂ is selected from the group consisting of arginine and lysine;
AA₁₃ is selected from the group consisting of glutamic acid, glutamine, aspartic acid, asparagine and an aliphatic, substituted aliphatic, benzyl, substituted benzyl, aromatic or substituted aromatic ester of glutamic acid or aspartic acid; and
AA₁₄ is selected from the group consisting of isoleucine, threonine, leucine, methionine, valine and serine.

24. The peptide of claim 23 wherein the sequence AA₁ through AA₁₄ or a subsequence thereof corresponds to a sequence of the B4-5 region of a cyclin dependent kinase selected from the group consisting of SEQ ID NO: 3 and SEQ ID NO: 4 or a subsequence thereof, with the proviso that any two amino acids in the sequence AA₁ through AA₁₄ or the subsequence thereof can vary.

25. The peptide of claim 23 wherein the sequence AA₁ through AA₁₄ or a subsequence thereof corresponds to a sequence of the B4-5 region of a cyclin dependent kinase selected from the group consisting of SEQ ID NO: 3 and SEQ ID NO: 4 or a subsequence thereof, with the proviso that any one amino acid in the sequence AA₁ through AA₁₄ or the subsequence thereof can vary.

26. A peptide comprising a sequence of amino acids AA₁ through AA₁₄ or a subsequence thereof comprising at least five amino acids, wherein:
AA₁ is selected from the group consisting of phenylalanine, tryptophan and tyrosine;
AA₂ is selected from the group consisting of leucine, isoleucine, methionine and valine;
AA₃ is selected from the group consisting of threonine, glycine, alanine, valine, serine, isoleucine, leucine and methionine;
AA₄ is selected from the group consisting of alanine, serine, glycine and threonine;
AA₅ is selected from the group consisting of glutamic acid, aspartic acid, glutamine, asparagine, and an aliphatic, substituted aliphatic, benzyl, substituted benzyl, aromatic or substituted aromatic ester of glutamic acid or aspartic acid;
AA₆ is selected from the group consisting of glutamic acid, lysine, methionine, isoleucine, asparagine, aspartic acid, glutamine, arginine, leucine, valine and an aliphatic, substituted aliphatic, benzyl, substituted benzyl, aromatic or substituted aromatic ester of glutamic acid or aspartic acid;
AA₇ is selected from the group consisting of arginine, threonine, lysine and serine;
AA₈ is selected from the group consisting of lysine, glycine, serine, aspartic acid, valine, arginine, alanine, threonine, glutamic acid, glutamine, asparagine, isoleucine, leucine, methionine and an aliphatic, substituted aliphatic, benzyl, substituted benzyl, aromatic or substituted aromatic ester of glutamic acid or aspartic acid;
AA₉ is selected from the group consisting of threonine, serine, arginine, asparagine, lysine, glutamine, glutamic acid, aspartic acid and an aliphatic, substituted aliphatic, benzyl, substituted benzyl, aromatic or substituted aromatic ester of glutamic acid or aspartic acid;
AA₁₀ is selected from the group consisting of glutamic acid, serine, asparagine, histidine, glysine, alanine, glutamine, aspartic acid, threonine and an aliphatic, substituted aliphatic, benzyl, substituted benzyl, aromatic or substituted aromatic ester of glutamic acid or aspartic acid;
AA₁₁ is selected from the group consisting of leucine, valine, serine, threonine, aspartic acid, isoleucine, methionine, glutamic acid, glutamine, asparagine and an aliphatic, substituted aliphatic, benzyl, substituted benzyl, aromatic or substituted aromatic ester of glutamic acid or aspartic acid;
AA₁₂ is selected from the group consisting of glycine, aspartic acid, glutamic acid, serine, tryptophan, alanine, glutamine, asparagine, threonine, phenylalanine, tyrosine and an aliphatic, substituted aliphatic, benzyl, substituted benzyl, aromatic or substituted aromatic ester of glutamic acid or aspartic acid;
AA₁₃ is selected from the group consisting of lysine, valine, threonine, arginine, isoleucine, leucine, methionine and serine; and
AA₁₄ is selected from the group consisting of methionine, isoleucine, leucine and valine.

27. The peptide of claim 26 wherein the sequence AA₁ through AA₁₄ or a subsequence thereof corresponds to the sequence of the B4-5 region of a TGF receptor type II SEQ ID NO: 5 or a subsequence thereof; an activin receptor type II selected from the group consisting of SEQ ID NO: 6 and SEQ ID NO: 7 or a subsequence thereof; an activin receptor-like kinase selected from the group consisting of SEQ ID NO: 8, SEQ ID NO: 9, SEQ ID NO: 10 and SEQ ID NO. 11 or a subsequence thereof; and bone morphogenetic protein receptor type II SEQ ID NO: 12 or a subsequence thereof, with the proviso that any two amino acids in the sequence AA₁ through AA₁₄ or the subsequence thereof can vary.

28. The peptide of claim 26 wherein the sequence AA₁ through AA₁₄ or a subsequence thereof corresponds to the sequence of the B4-5 region of a TGF receptor type II SEQ ID NO: 5 or a subsequence thereof; an activin receptor type II selected from the group consisting of SEQ ID NO: 6 and SEQ ID NO: 7 or a subsequence thereof, an activin receptor-like kinase selected from the group consisting of SEQ ID NO: 8, SEQ ID NO: 9, SEQ ID NO: 10 and SEQ ID NO. 11 or a subsequence thereof; and bone morphogenetic protein receptor type II SEQ ID NO: 12 or a subsequence thereof, with the proviso that any one amino acid in the sequence AA₁ through AA₁₄ or the subsequence thereof can vary.

29. A peptide comprising a sequence of amino acids AA₁ through AA₂₃ or a subsequence thereof comprising at least five amino acids, wherein:
AA₁ is selected from the group consisting of isoleucine, valine, leucine and methionine;
AA₂ is selected from the group consisting of arginine, asparagine, phenylalanine, lysine, aspartic acid, glutamic acid, glutamine, tryptophan, tyrosine and an aliphatic, substituted aliphatic, benzyl, substituted benzyl, aromatic or substituted aromatic ester of glutamic acid or aspartic acid;
AA₃ is selected from the group consisting of aspartic acid, glutamic acid, glutamine, asparagine and an aliphatic, substituted aliphatic, benzyl, substituted benzyl, aromatic or substituted aromatic ester of glutamic acid or aspartic acid;
AA₄ is selected from the group consisting of isoleucine, leucine, methionine and valine;
AA₅ is selected from the group consisting of leucine, isoleucine, methionine and valine;
AA₆ is selected from the group consisting of arginine, glycine, lysine and alanine;
AA₇ is selected from the group consisting of alanine, proline and glycine;
AA₈ is selected from the group consisting of proline, serine and threonine;
AA₉ is selected from the group consisting of threonine, glycine, serine and alanine;
AA₁₀ is selected from the group consisting of leucine, isoleucine, serine, methionine, valine and threonine;
AA₁₁ is selected from the group consisting of glutamic acid, glutamine, aspartic acid, asparagine and an aliphatic, substituted aliphatic, benzyl, substituted benzyl, aromatic or substituted aromatic ester of glutamic acid or aspartic acid;
AA₁₂ is selected from the group consisting of alanine, glutamine, leucine, glycine, glutamic acid, aspartic acid, asparagine, isoleucine, methionine, valine and an aliphatic, substituted aliphatic, benzyl, substituted benzyl, aromatic or substituted aromatic ester of glutamic acid or aspartic acid;
AA₁₃ is selected from the group consisting of methionine, threonine, isoleucine, leucine, valine and serine;
AA₁₄ is selected from the group consisting of arginine, lysine, aspartic acid, glutamic acid, glutamine, asparagine and an aliphatic, substituted aliphatic, benzyl, substituted benzyl, aromatic or substituted aromatic ester of glutamic acid or aspartic acid;
AA₁₅ is selected from the group consisting of aspartic acid, glutamic acid, glutamine, asparagine and an aliphatic, substituted aliphatic, benzyl, substituted benzyl, aromatic or substituted aromatic ester of glutamic acid or aspartic acid;
AA₁₆ is selected from the group consisting of valine, isoleucine, leucine and methionine;
AA₁₇ is selected from the group consisting of glycine and alanine;
AA₁₈ is selected from the group consisting of serine and threonine;
AA₁₉ is selected from the group consisting of leucine, isoleucine, methionine and valine;
AA₂₀ is selected from the group consisting of threonine and serine;
AA₂₁ is selected from the group consisting of glutamic acid, aspartic acid, glutamine, asparagine and an aliphatic, substituted aliphatic, benzyl, substituted benzyl, aromatic or substituted aromatic ester of glutamic acid or aspartic acid;
AA₂₂ is selected from the group consisting of leucine, isoleucine, methionine and valine; and
AA₂₃ is selected from the group consisting of asparagine, glutamine, glutamic acid, aspartic acid and an aliphatic, substituted aliphatic, benzyl, substituted benzyl, aromatic or substituted aromatic ester of glutamic acid or aspartic acid.

30. The peptide of claim 29 of the sequence AA₁ through AA₂₃ or a subsequence thereof corresponds to the sequence of the B4-5 region of an extra-cellular signal-regulated kinase selected from the group consisting of SEQ ID NO: 13, SEQ ID NO: 14 and SEQ ID NO: 15 or a subsequence thereof, with the proviso that any two amino acids in the sequence AA₁ through AA₂₃ or the subsequence thereof can vary.

31. The peptide of claim 29 wherein the sequence AA₁ through AA₂₃ or a subsequence thereof corresponds to the sequence of the B4-5 region of an extra-cellular signal-regulated kinase selected from the group consisting of SEQ ID NO: 13, SEQ ID NO: 14 and SEQ ID NO: 15 or a subsequence thereof, with the proviso that any one amino acid in the sequence AA₁ through AA₂₃ or the subsequence thereof can vary.

32. A peptide comprising a sequence of amino acids AA₁ through AA₁₅ or a subsequence thereof comprising at least five amino acids, wherein:
AA₁ is selected from the group consisting of leucine, isoleucine, methionine and valine;
AA₂ is selected from the group consisting of leucine, isoleucine, methionine and valine;
AA₃ is selected from the group consisting of asparagine, glutamine, glutamic acid, aspartic acid and an aliphatic, substituted aliphatic, benzyl, substituted benzyl, aromatic or substituted aromatic ester of glutamic acid or aspartic acid;
AA₄ is selected from the group consisting of valine, isoleucine, leucine and methionine;
AA₅ is selected from the group consisting of phenylalanine, tryptophan and tyrosine;
AA₆ is selected from the group consisting of threonine and serine;
AA₇ is proline;
AA₈ is selected from the group consisting of glutamine, aspartic acid, glutamic acid, asparagine and an aliphatic, substituted aliphatic, benzyl, substituted benzyl, aromatic or substituted aromatic ester of glutamic acid or aspartic acid;
AA₉ is selected from the group consisting of lysine and arginine;
AA₁₀ is selected from the group consisting of serine and threonine;
AA₁₁ is selected from the group consisting of leucine, isoleucine, methionine and valine;
AA₁₂ is selected from the group consisting of glutamic acid, glutamine, aspartic acid, asparagine and an aliphatic, substituted aliphatic, benzyl, substituted benzyl, aromatic or substituted aromatic ester of glutamic acid or aspartic acid;
AA₁₃ is selected from the group consisting of glutamic acid, glutamine, aspartic acid, asparagine and an aliphatic, substituted aliphatic, benzyl, substituted benzyl, aromatic or substituted aromatic ester of glutamic acid or aspartic acid;
AA₁₄ is selected from the group consisting of phenylalanine, tryptophan and tyrosine; and
AA₁₅ is selected from the group consisting of glutamine, asparagine, glutamic acid, aspartic acid and an aliphatic, substituted aliphatic, benzyl, substituted benzyl, aromatic or substituted aromatic ester of glutamic acid or aspartic acid.

33. The peptide of claim 32 wherein the sequence AA₁ through AA₁₅ or a subsequence thereof corresponds to the sequence of the B4-5 region of a Jun N-terminal kinase selected from the group consisting of SEQ ID NO: 16 and SEQ ID NO: 17 or a subsequence thereof, with the proviso that any two amino acids in the sequence AA₁ through AA₁₅ or the subsequence thereof can vary.

34. The peptide of claim 32 wherein the sequence AA₁ through AA₁₅ or a subsequence thereof corresponds to the sequence of the B4-5 region of a Jun N-terminal kinase selected from the group consisting of SEQ ID NO: 16 and SEQ ID NO: 17 or a subsequence thereof, with the proviso that any one amino acid in the sequence AA₁ through AA₁₅ or the subsequence thereof can vary.

35. A method of identifying a peptide which modulates the activity of a protein kinase comprising the steps of:
a) providing a peptide, referred to as a “test peptide”, comprising a peptide derivative of the B4-5 region of said protein kinase and having from about five to about twenty-five amino acids or analogs thereof;
b) incubating the test peptide with cells having one or more cellular activities controlled by a protein kinase under conditions suitable for assessing activity of the protein kinase;
c) assessing activity of the protein kinase, wherein greater or lesser activity compared with the cells grown without incubation of the test peptide indicates that the peptide modulates activity of the protein kinase.

36. The method of claim 35, wherein the activity of the protein kinase is assessed by measuring the rate of survival or proliferation of said cells in tissue culture.

37. A method of modulating the cellular activity of a protein kinase in a subject, comprising administering a therapeutically effective amount of a peptide comprising a peptide derivative of the B4-5 region of a protein kinase, wherein:
a) said peptide has between about five and about twenty-five amino acids or amino acid analogs; and
b) said peptide modulates cellular activity of the protein kinase.

38. A method of detecting a ligand that binds to the B4-5 region of a protein kinase comprising:
a) providing a peptide derivative of the B4-5 region of said protein kinase, said peptide derivative having at least five amino acids or analogs thereof;
b) incubating said peptide derivative with a sample, to be tested for the presence of said ligand, for a time sufficient for said ligand to bind to said peptide derivative; and
c) detecting any said ligand-said peptide derivative binding pair that has been formed in step b), wherein the presence of said ligand-said peptide derivative binding pair establishes the existence of said ligand in said sample.

39. The method of claim 38 further comprising:
d) separating said ligand from said peptide derivative; and
e) determining the structure of said ligand, thereby identifying said ligand.

40. An antibody that immunologically binds to the B4-5 region of a protein kinase.

41. A method of producing antibodies that bind to the B4-5 region of a protein kinase comprising:
a) providing a peptide derivative of the B4-5 region of said protein kinase, said peptide derivative having at least five amino acids; and
b) producing antibodies to said peptide derivative.
